(19) United States
(12) Patent Application Publication
(10) Pub. No.: US 2011/0275675 A1
(43) Pub. Date: Nov. 10, 2011

(54) Antimicrobial Dental Materials

(75) Inventors: Kai Rist, Feldkirch (AT); Ulrich Salz, Lindau (DE); Volker Rheinberger, Vaduz (LI)

(73) Assignee: IVOCLAR VIVADENT AG, Schaan (LI)

(21) Appl. No.: 13/097,100

(22) Filed: Apr. 29, 2011

(30) Foreign Application Priority Data
May 7, 2010 (EP) 10162250.4

(51) Int. Cl. A01N 43/40; A01P 1/00; C07D 401/06

(52) U.S. Cl. 514/332; 546/264

(57) ABSTRACT

Dental material which contains an antimicrobial active ingredient according to general formula (I),

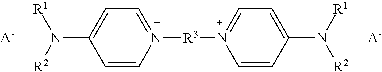

in which R¹=H, C₁ to C₃ alkylene residue; R²=a linear or branched C₄ to C₂₀ alkylene residue; R³=a C₆ to C₂₀ alkylene residue; and A⁻=Cl⁻, Br⁻, I⁻, F⁻, OH⁻, alkyl and aryl alcoholate, thiolate, NO₃⁻, BO₃³⁻, PO₄³⁻, HPO₄²⁻, H₂PO₄⁻, alkyl and aryl phosphate, alkyl and aryl phosphonate, PF₆⁻, TiF₆²⁻, BF₄⁻, acetylacetonate, β-diketonate or anion of a β-keto ester. The dental material is suitable in particular as tooth filling material, material for inlays or onlays, tooth cement, enamel/dentine adhesive, veneering material for crowns and bridges, or as material for false teeth.

### CROSS REFERENCE TO RELATED APPLICATIONS

This application claims the benefit of European Patent Application No. 10162250.4, filed May 7, 2010, which is hereby incorporated by reference in its entirety.

### TECHNICAL FIELD

The present invention relates to dental materials with antimicrobial action which are suitable inter alia as filling materials, cements, sealants, materials for temporary restorations, coating materials and adhesives.

### BACKGROUND OF THE INVENTION

Most dental treatments become necessary when pathogenic germs colonize the dentine and enamel, the marginal gaps between the dentine and enamel and dental restorations, restoration and prosthetic materials as well as the neighbouring soft tissue. In particular, the bacteria Streptococcus mutans and lactobacilli produce acids as metabolic products which, as in the case of extensive caries, cause immense hard tissue damage as a result of tissue decalcification and enzymatic collagen degradation. Thus for example when filling a root canal with a known inert filling material it cannot be ruled out that an inflammatory process will slowly result after the filling as a result of germs that have remained in the canal, which makes a renewed treatment necessary, which then very often leads to the tooth being lost entirely. Prosthesis incompatibilities are frequently attributable to the colonization of the prosthesis surfaces by Candida albicans, a fungus of the Candida group.

Mouthwashes, toothpastes and gels which contain an antimicrobial active ingredient such as e.g. triclosan or chlorhexidine are used for example for the prevention and treatment of caries in order to suppress pathogenic, intraoral microbes. While chlorhexidine is one of the most effective means of controlling plaque and inflammation of the gums, triclosan has the advantage that it is non-ionic and therefore compatible with most constituents of oral hygiene agents. Triclosan is therefore used in most commercially available products. A disadvantage of these forms of administration is the short impact time. This makes a protracted, repeated application indispensable in order to achieve the desired treatment result, which in turn requires a costly aftercare. When colonies of pathogenic germs are difficult or even impossible to access, such as in deep fissures, approximal spaces or marginal gaps, this form of treatment is unsuitable. When bacteria colonize marginal gaps, in most cases the only practical measure is to replace the filling.

Antimicrobially-acting dental materials are frequently used to preclude these destructive treatments. The antimicrobial action is most often achieved by adding antimicrobial active ingredients to the dental material. As a rule, the same active ingredients are used as are also used in mouthwashes, toothpastes or gels. A problem is that the physical and chemical properties of the dental material, such as its mechanical properties or the hardening behaviour, must not be impaired by the addition of active ingredient. A release of the active ingredient in an effective quantity and over an extended, clinically relevant timespan must also be ensured.

A chlorhexidine-containing dental cement is described in WO 89/10736 which is very soluble in oral fluids and is said to dissolve after one to four weeks. A controlled release of the active ingredient is said to be thereby achieved.

DE 19813686 A1 and U.S. Pat. No. 6,162,056, which is hereby incorporated by reference, discloses an antimicrobially-acting root filling material which contains chlorhexidine and an iodophor. The active ingredient is applied to a polymeric carrier.

An antimicrobial etching gel based on phosphoric acid is described in U.S. Pat. No. 5,385,728, which is hereby incorporated by reference, which contains benzalkonium chloride in a preferred concentration range from 0.1 to 4.0 wt.-%.

WO 99/20227 and US2001009931, which is hereby incorporated by reference, describes a tooth-coating material for the prevention of caries which contains triclosan (2,4,4′-trichloro-2′-hydroxydiphenylether) as antimicrobial active ingredient.

Curable dental restoration materials are described in US 2003/0220416 A1, which is hereby incorporated by reference, which contain water-insoluble, non-cationic antibacterial substances, preferably triclosan.

EP 1566160 A2 and US 2005180930, which is hereby incorporated by reference, discloses the use of extracts from natural products with antimicrobial action, such as for example grapefruit seed extract or green tea extract, to prepare dental materials. The extracts are included in cyclodextrins.

WO 2005/058252, which is hereby incorporated by reference, discloses the use of elemental silver as antibacterial active ingredient for dental materials, and EP 1374830 A1 and U.S. Pat. No. 6,924,325, which is hereby incorporated by reference, describes a dental composition with antimicrobial properties which contains silver-containing ceramics.

However, each of these listed active ingredients has suffered from negative properties which greatly limit their use in dental materials and which call into question the clinical suitability of these materials. For example, active ingredients such as triclosan inhibit radical polymerization, which impairs the mechanical properties of polymerizable dental materials and restricts the usable quantity of active ingredient. Other active ingredients, such as e.g. chlorhexidine, lead to a clear blackening of teeth, tongue and fillings when administered orally over an extended period (Gjermo P J, Dent Res, Spec Iss 1989; 68:1602-1608). In addition, because of its bitter taste chlorhexidine leads to taste irritations and is not light-stable. When exposed to sunlight, the toxicologically problematical p-chloroaniline forms (Kohlbecker G, Dtsch Zahnärztl Z 1989; 44: 273-276). Silver also leads to discolorations in the oral cavity through the formation of black silver sulphide or yellow-coloured silver phosphate. In addition, the effective range of the named active ingredients is often insufficient.

### SUMMARY OF THE INVENTION

The object of the invention is to provide dental materials which are characterized by a high antimicrobial action and which do not display the named disadvantages.

### BRIEF DESCRIPTION OF THE DRAWINGS

The invention is described in further detail in the following with reference the drawings.

FIG. 1 shows zones of inhibition which form on agar plates inoculated with S. mutans around small plates made of a dental material according to the invention. In contrast, zones of inhibition do not form around small plates made of dental material without active ingredient. The test shows that active ingredients of formula (I) are released in an effective quantity from polymerized dental materials.

FIG. 2 shows an enlarged section from FIG. 1.

FIG. 3 shows small plates of a hardened comparison example which contains triclosan in a concentration of 1.0 wt.-% on an agar plate inoculated with S. mutans. Unlike with the small plates made of a dental material according to the invention, zones of inhibition do not form around small plates made of dental material with triclosan as active ingredient.

FIG. 4 shows small plates of a hardened comparison example which contains triclosan in a concentration of 3.0 wt.-% on an agar plate inoculated with S. mutans. Unlike with the small plates made of a dental material according to the invention, zones of inhibition do not form around small plates made of dental material with triclosan as active ingredient.

### DETAILED DESCRIPTION

The object is achieved according to the invention by dental materials which contain an antimicrobial active ingredient from the group of bis(4-amino-1-pyridinium) salts according to general formula (I):

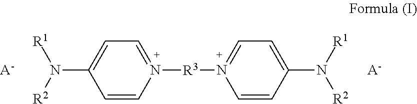

in which the variables R¹, R², R³ and A⁻, independently of each other, have the following meanings:
R¹=H, a C₁ to C₃ alkyl residue, preferably H;
R²=a C₆ to C₁₆ alkyl, cycloalkyl or alkyl aryl residue, preferably a C₈ to C₁₂ alkyl residue;
R³=a C₆ to C₂₀ alkylene residue, preferably C₈ to C₁₄ residue;
A⁻=Cl⁻, Br⁻, I⁻, F⁻, alkyl and aryl alcoholate, thiolate, NO₃⁻, BO₃³⁻, PO₄³⁻, HPO₄²⁻, H₂PO₄⁻, alkyl and aryl phosphate, alkyl and aryl phosphonate, PF₆⁻, TiF₆²⁻, BF₄⁻, acetylacetonate, β-diketonate, anion of a β-keto ester.

The antimicrobial active ingredient according to formula (I) in which the variables have the following meanings is particularly preferred:

### R¹=H

R²=a linear C₈ alkyl residue
R³=a linear C₁₀ alkylene residue
A⁻=Cl⁻, alkyl phosphate, alkyl phosphonate.

This active ingredient has the following formula (Ia) and is commercially available.

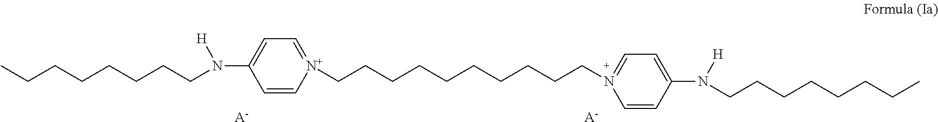

The antimicrobial active ingredients of formula (I) according to the invention can be prepared by known synthesis processes, such as are described e.g. in U.S. Pat. No. 4,206,215, which is hereby incorporated by reference.

It was found that the active ingredients of formula (I) have a combination of properties that is advantageous for preparing dental materials. On the one hand they are lipophilic enough to be compatible with organic monomers, on the other hand they have such a high water-solubility that they can exert their antimicrobial action in full. Moreover, it was found that they do not inhibit radical polymerization.

The dental materials according to the invention are suitable in particular as filling materials and temporary filling materials, e.g. for prepared cavities and root canals, as underfilling materials, as cements and temporary cements, for example for cementing indirect restorations, as sealants for pits, fissures, root canals, root, dentine and enamel surfaces, as temporary restoration materials, e.g. as materials for temporary crowns and bridges, as adhesives, as coating materials, etchants, and root canal sealants.

Preferred dental materials are:

Adhesives, such as enamel/dentine adhesives which require a substrate conditioning with an acid prior to the application of adhesive (total etch technique); self-etching enamel/dentine adhesives.
Temporary restoration materials, such as composite-based temporary fixing cements; temporary fixing cements based on polycarboxylate/zinc oxide; composite-based temporary filling materials; temporary filling materials based on glass ionomer; temporary filling materials based on polycarboxylate/zinc oxide; temporary composite- or PMMA-based crown and bridge materials.
Cements, such as glass ionomer cements, glass ionomer hybrid materials, polycarboxylate cements.
Filling materials, such as compomer filling materials, composite filling materials, composite fixing materials, bracket cements.
Sealants, such as fissure sealants and acrylate coating materials.

The dental materials according to the invention preferably contain 0.01-3.0 wt.-%, particularly preferably 0.05-1.5 wt.-% and quite particularly preferably 0.1-0.6 wt.-% of the active ingredient according to Formula (I). Unless stated otherwise, all percentages relate to the total composition of the dental materials.

According to the invention, polymerizable dental materials are particularly preferred, in particular dental materials which can be cured by a radical polymerization. These contain, in addition to the active ingredient according to formula (I), at least one polymerizable, ethylenically unsaturated monomer as binder, an initiator for the radical polymerization and can also contain an organic and/or inorganic filler.

The polymerizable dental materials are particularly suitable

as tooth filling materials, materials for inlays or onlays, tooth cements, as veneering materials for crowns and bridges, as material for false teeth, materials for temporary restorations or as other materials for prosthetic, restorative and preventative dentistry,
as enamel/dentine adhesives,
coating material for tooth fillings, restoration materials, dentures and natural teeth.

Amorphous, spherical particles of silicon dioxide and up to 20 mol-% of an oxide of at least one element of groups I, II, III and IV of the periodic table are particularly suitable as inorganic fillers. These preferably have a refractive index of 1.50 to 1.58 and an average primary particle size of 0.1 to 1.0 μm.

Other preferred fillers are quartz, glass ceramic or glass powders or mixtures thereof. These preferably also have a refractive index of 1.50 to 1.58 and an average particle size of 0.4 to 5.0 μm.

Filler mixtures which contain at least two different fillers are particularly preferably used. Filler mixtures which contain at least one filler based on the above spherical silicon dioxide particles as first filler component and at least one quartz, glass ceramic and/or glass powder as second filler component are preferred.

The amorphous, spherical silicon dioxide particles are an amorphous, spherical material based on silicon dioxide which contains, in addition, an oxide of at least one metal of groups I, II, III and IV of the periodic table. Strontium and/or zirconium oxide is preferably used. The average primary particle size is in the range from 0.1 to 1.0 μm, in particular 0.15 to 0.5 μm. The refractive index of this inorganic filler is preferably between 1.50 and 1.58, in particular between 1.52 and 1.56. A particularly preferred value is 1.53±0.01. Mixtures of different particles can also be used. Fillers of this type are described in DE-PS 32 47 800. The filler particles can also be present sintered as a mixture of agglomerates with an average particle size of 1 to 30 μm.

Equally preferred fillers are quartz, glass ceramic and, in particular, glass powders. The average particle size of this inorganic filler is to be between 0.5 and 5.0 μm, in particular between 1.0 and 2.0 μm and particularly preferably between 1.0 and 1.5 μm, while the refractive index is to have values between 1.50 and 1.58, in particular between 1.52 and 1.56. Filler mixtures can also be used. According to the invention, Ba-silicate glasses with an average particle size in the range from 1.1 to 1.3 μm, and also Sr-silicate glasses with an average particle size in the range from 1.1 to 1.3 μm, and also Li/Al-silicate glasses with an average particle size of 1.0 to 1.6 μm are preferably used. Such powders can be obtained e.g. by fine grinding with an RS ultrafine mill from Reimbold & Strich, Cologne.

Optionally, in addition to the first and/or second filler component, still further fillers can be used to achieve an increased X-ray opacity, wherein their average primary particle size should not exceed 5.0 μm. Such fillers are described e.g. in DE-OS 35 02 594 and U.S. Pat. No. 4,629,746, which is hereby incorporated by reference. A particularly preferably used further filler is ytterbium trifluoride.

Optionally, small quantities of microfine, pyrogenic or wet-precipitated silicic acid can be incorporated into the dental material to adjust the viscosity, preferably however at most 5 wt.-%, relative to the dental material.

The total filler quantity, consisting of the above-named fillers in the dental material according to the invention, is between 0 and 90 wt.-%, preferably 2 to 85 wt.-% and particularly preferably 10 to 80 wt.-% depending on the intended use. The weight percentage of the first filler component is preferably 5-60%, in particular 10-30%, the percentage of the second filler component 15-85%, in particular 30-70%, in each case relative to the total dental material.

The inorganic fillers are preferably silanized. α-methacryloxypropyltrimethoxysilane e.g. is suitable as adhesion promoter. The quantity of adhesion promoter used is based on the type and BET surface area of the filler.

All binders that can be used for a dental material are suitable as polymerizable organic binders, in particular monofunctional or polyfunctional (meth)acrylates or (meth)acrylamides which can be used alone or in mixtures. Coming into consideration as examples of these compounds are methyl methacrylate, 2-hydroxyethyl (meth)acrylate, 2-hydroxypropyl (meth)acrylate, isobutyl (meth)acrylate, cyclohexyl (meth)acrylate, glycerol di(meth)acrylate, tetraethyleneglycol di(meth)acrylate, triethyl-leneglycol di(meth)acrylate, diethyleneglycol di(meth)acrylate, ethylene glycol di(meth)acrylate, polyethylene glycol di(meth)acrylate, butanediol di(meth)acrylate, hexanediol di(meth)acrylate, decanediol di(meth)acrylate, dodecanediol di(meth)acrylate, bisphenol A di(meth)acrylate, trimethylolpropane tri(meth)acrylate, 2,2-bis-4(3-(meth)acryloxy-2-hydroxy-propoxy)-phenylpropane (bis-GMA) and also the reaction products of isocyanates, in particular di- and/or triisocyanates, and OH-group-containing (meth)acrylates. Examples of this are the reaction products of 1 mol hexamethylene diisocyanate with 2 mol 2-hydroxyethylene methacrylate, of 1 mol tri(6-isocyanatohexyl)biuret with 3 mol 2-hydroxyethyl methacrylate and of 1 mol 2,2,4-trimethylhexamethylene diisocyanate with 2 mol 2-hydroxyethyl methacrylate, which are called urethane dimethacrylates in the following.

N-mono- or N-disubstituted acrylamides, such as e.g. N-ethylacrylamide, N,N-dimethylacrylamide, N-(2-hydroxy-ethyl)acrylamide or N-methyl-N-(2-hydroxyethyl)acrylamide, and N-monosubstituted methacrylamides, such as e.g. N-ethylmeth-acrylamide or N-(2-hydroxyethyl)methacrylamide and also N-vinylpyrrolidone and allyl ether. These monomers are liquid at room temperature and are therefore also suitable as diluent monomers.

Monomers with two or more radically polymerizable groups are suitable as cross-linking monomers, in particular cross-linking pyrrolidones, such as e.g. 1,6-bis-(3-vinyl-2-pyrrolidonyl)-hexane, or commercially available bisacrylamides such as methylene or ethylene bisacrylamide, bis(meth)acrylamides, such as e.g. N,N′-diethyl-1,3-bis(acrylamido)propane, 1,3-bis(methacrylamido)propane, 1,4-bis(acrylamido)butane or 1,4-bis(acryloyl)piperazine, which can be synthesized by reaction from the corresponding diamines with (meth)acrylic acid chloride.

The dental materials according to the invention preferably contain at least one cross-linking monomer. The percentage of cross-linking monomers in the dental material preferably ranges from 10 to 95 wt.-%, preferably 20 to 75 wt.-%.

The dental materials according to the invention can also contain a radically polymerizable, acid-group-containing monomer. Acid-group-containing monomers are also called acidic monomers in the following. The dental materials preferably contain a mixture of acidic and non-acidic monomers. Preferred acid groups are carboxylic acid groups, phosphonic acid groups, phosphate groups and/or sulphonic acid groups, wherein these groups can be present in acid form or in the form of an ester. Monomers with phosphonic acid groups or phosphate groups are preferred. The monomers can have one or more acid groups, compounds with 1 to 2 acid groups being preferred.

Preferred polymerizable carboxylic acids are maleic acid, acrylic acid, methacrylic acid, 2-(hydroxymethyl)acrylic acid, 4-(meth)acryloyloxyethyltrimellic acid and the corresponding anhydride, 10-methacryloyloxydecylmalonic acid, N-(2-hydroxy-3-methacryloyloxypropyl)-N-phenylglycine and 4-vinylbenzoic acid.

Preferred phosphonic acid monomers are vinyl phosphonic acid, 4-vinylphenyl phosphonic acid, 4-vinylbenzyl phosphonic acid, 2-methacryloyloxyethyl phosphonic acid, 2-methacrylamidoethyl phosphonic acid, 4-methacrylamido-4-methyl-pentyl-phosphonic acid, 2-[4-(dihydroxyphosphoryl)-2-oxa-butyl]-acrylic acid and 2-[2-dihydroxyphosphoryl)-ethoxymethyl]-acrylic acid-2,4,6-trimethylphenyl ester.

Preferred acidic polymerizable phosphoric acid esters are 2-methacryloyloxypropyl mono- and dihydrogenphosphate, 2-methacryloyloxyethyl mono- and dihydrogenphosphate, 2-methacryloyloxyethyl-phenyl-hydrogenphosphate, dipentaerythritol-pentamethacryloyloxyphosphate, 10-methacryloyloxydecyl dihydrogen phosphate, dipentaerythritolpentamethacryloyloxyphosphate, phosphoric acid mono-(1-acryloyl-piperidin-4-yl)-ester, 6-(methacrylamido)hexyl dihydrogen phosphate and 1,3-bis-(N-acryloyl-N-propylamino)-propan-2-yl-dihydrogenphoshate.

Preferred polymerizable sulphonic acids are vinylsulphonic acid, 4-vinylphenylsulphonic acid or 3-(methacrylamido)-propylsulphonic acid.

The percentage of acidic monomers in the dental material ranges from 0 to 85 wt.-%, preferably 0 to 60 wt.-%.

Moreover, the dental materials according to the invention can also contain solvents, preferably 0 to 80 wt.-%, particularly preferably 0 to 60 wt.-% and quite particularly preferably 0 to 40 wt.-% solvent.

Solvents are mainly used in adhesives and coating materials. Preferred solvents are water, methanol, ethanol, phenoxyethanol, isopropanol, ethyl acetate, acetone and mixtures thereof.

Depending on the type of initiator used, the dental material can be hot-, cold- or light-polymerizable. The known peroxides such as dibenzoyl peroxide, dilauroyl peroxide, tert-butyl peroctoate or tert-butyl perbenzoate can be used as initiators for the hot polymerization, but α,α′-azo-bis(isobutyroethylester), benzopinacol and 2,2′-dimethylbenzopinacol are also suitable.

Benzophenone and its derivatives and also benzoin and its derivatives e.g. can be used as initiators for the photopolymerization. Further preferred photosensitizing agents are the α-diketones such as 9,10-phenanthrenequinone, diacetyl, furil, anisil, 4,4′-dichlorobenzil and 4,4′-dialkoxybenzil. Camphorquinone is particularly preferably used. The use of the photosensitizing agents together with a reducing agent is preferred. Examples of reducing agents are amines such as cyanoethylmethylaniline, dimethylaminoethylmethacrylate, triethylamine, triethanolamine, N,N-dimethylaniline, N-methyldiphenylamine, N,N-dimethyl-sym.-xylidine and N,N-3,5-tetramethylaniline and 4-dimethylaminobenzoic acid ethyl ester. Furthermore, photoinitiators of the product group of the acylphosphine oxides, bisacylphosphine oxides or also acylgermanium compounds can be used.

Radical-generating systems, e.g. benzoyl or lauroyl peroxide are used as initiators for the cold polymerization together with amines such as N,N-dimethyl-sym.-xylidine or N,N-dimethyl-p-toluidine. Dual-curing systems can also be used for the initiation, e.g. photoinitiators with amines and peroxides. Mixtures of UV light curing and visible light curing initiators also come into consideration as photoinitiators.

The quantity of these initiators in the dental material is usually between 0.01 and 5 wt.-%, preferably 1 to 3.5 wt.-%.

Fine-particle splinter or bead polymerisates which can be homo- or copolymers of the already described vinyl compounds can also be incorporated into the dental material. These homo- or copolymers can in turn be filled with the described inorganic fillers, also X-ray opaque. The dental material can further contain the usual pigmenting agents and stabilizers.

The dental materials according to the invention preferably have the following overall composition:

about 0.01-about 3.0 wt.-%, preferably about 0.05-about 1.5 wt.-% and particularly preferably about 0.1-about 0.6 wt.-% active ingredient according to formula (I);
about 20 to about 95 wt.-%, preferably about 30 to about 90 wt.-% and particularly preferably about 35 to about 80 wt.-% binder,
about 0.1 to about 5 wt.-%, preferably about 0.3 to about 4.0 wt.-% and particularly preferably about 0.5 to about 3.5 wt.-% initiator; and optionally
0 to about 90 wt.-%, preferably about 2 to about 85 wt.-% and particularly preferably about 10 to about 80 wt.-% filler; and optionally
0 to about 80 wt.-%, particularly preferably 0 to about 60 wt.-% and quite particularly preferably 0 to about 40 wt.-% solvent.

The dental materials according to the invention are suitable for example for preventing tooth damage caused by Streptococcus mutans and/or lactobacilli or also for preventing prosthesis incompatibilities caused by Candida albicans.

The invention is described in further detail with reference to the following nonlimiting examples.

### Example 1

### Antimicrobial Enamel/Dentine Adhesives

To study the influence of the antimicrobial active ingredient according to formula (Ia) on the dentine and enamel adhesion of adhesive formulations, mixtures of the following composition were prepared:

### Example 1a

### Antimicrobial Enamel/Dentine Adhesive for the Total Etch Technique

(i) 17.1 wt.-% of the cross-linking agent glycerol dimethacrylate (GDMA);
(ii) 69 wt.-% of the hydrophilic monomer 2-hydroxyethyl methacrylate (HEMA);
(iii) 12 wt.-% ethanol;
(iv) 1.0 wt.-% of the antimicrobial active ingredient according to formula (Ia);
(v) 0.5 wt.-% ethyl-p-dimethylaminobenzoate;
(vi) 0.4 wt.-% camphorquinone.

In the case of the non-antimicrobial comparison adhesive, the antimicrobial active ingredient was replaced by the corresponding quantity of GDMA.

### Adhesion Value Studies (Shearing Adhesion Strength)

To measure dentine or enamel adhesion, bovine teeth cast in resin (Bühler Castolite resin) were used. Firstly, the cast-in bovine tooth was ground down approximally to the dentine or enamel with sandpaper of grit size P500. After a brief fine grinding with sandpaper of grit size P1000 and thorough washing with water, the tooth was prepared for the preparation of testpieces.

### Preparation of Testpieces

Phosphoric acid etching gel (Email Preparator GS, Ivoclar Vivadent AG) was applied to the dentine or enamel surface and washed with water after an action time of 30 seconds. The thus-conditioned surface was lightly patted dry with a paper towel and then the adhesive formulation was applied. After an action time of 10 seconds, the adhesive layer was polymerized for 20 seconds by irradiation with a polymerization light (Astralis® 7). The thus-prepared tooth was clamped in a shear adhesion mould, a filling composite applied in two layers (a max. total of 3 mm) by means of a round Delrin mould 4 mm in diameter and in each case exposed to light for 40 seconds with a polymerization light (Astralis® 7 from Ivoclar Vivadent AG; >500 mW/cm²). The thus-prepared testpiece was removed from the mould and immediately placed in water. After storage of the testpieces at 37° C. for 24 h, the shearing adhesion strength was determined with a universal testing machine (Zwick Z010) and a testpiece device (guillotine) at a rate of 0.8 mm/min. The adhesion values achieved are listed in Table 1.

### Example 1b

### Self-Etching, Antimicrobial Enamel/Dentine Adhesive

(i) 20 wt.-% of the acid monomer methacryloyloxydecyl dihydrogen phosphate (MDP);
(ii) 20 wt.-% of the acid monomer 4-methacryloyloxyethyl trimellitate anhydride (4-META);
(iii) 28.1 wt.-% of the cross-linking monomer urethane dimethacrylate (UDMA);
(iv) 29.3 wt.-% of a 1:1 acetone/water mixture;
(v) 1.0 wt.-% of the antimicrobial active ingredient of formula (Ia);
(vi) 0.4 wt.-% of the photoinitiator camphorquinone;
(vii) 0.5 wt.-% of the co-initiator ethyl-p-dimethylaminobenzoate;
(viii) 0.7 wt.-% of the photoinitiator 2,4,6-trimethylbenzoyldiphenylphosphine oxide.

In the case of the non-antimicrobial comparison adhesive, the antimicrobial active ingredient was replaced by the corresponding quantity of UDMA.

The determination of the adhesion value of this self-etching antimicrobial enamel/dentine adhesive was carried out analogously to example 1a, but with the difference that the substrate was not conditioned with phosphoric acid prior to the application of the adhesive. The adhesion values achieved are listed in Table 1.

As can be seen from Table 1, the addition of the antimicrobial active ingredient of formula (Ia) does not have a negative effect on the dentine or enamel adhesion properties of the adhesives.

TABLE 1
Dentine and enamel adhesion values of
the adhesives from examples 1a-1c

| | Dentine adhesion | Enamel adhesion |
|---|---|---|
| Adhesive | (MPa) | (MPa) |
| Ex. 1a | 17.9 ± 3.3 | 19.8 ± 4.7 |
| Comparison with ex. 1a*) | 19.1 ± 5.2 | 20.0 ± 4.6 |
| Ex. 1b | 23.2 ± 3.2 | 20.1 ± 4.1 |
| Comparison with ex. 1b*) | 21.9 ± 5.2 | 19.8 ± 5.2 |

*)contains no antimicrobial active ingredient

### Example 2

### Antimicrobial Bonding Material

The following composition was prepared by mixing the components:
(i) 50.1 wt.-% bis-GMA;
(ii) 49.1 wt.-% TEGDMA;
(iii) 0.1 wt.-% of the antimicrobial active ingredient of formula (Ia);
(iv) 0.3 wt.-% camphorquinone;
(v) 0.4 wt.-% of the co-initiator Genocure EPD;

After 20 s exposure to light with a dental polymerization light (Bluephase® C8, Ivoclar Vivadent AG), the bonding material hardened completely.

### Example 3

### Antimicrobial Temporary Fixing Cement Based on Polycarboxylate/Zinc Oxide

0.3 wt.-% or 3.0 wt.-% of the antimicrobial active ingredient of formula (Ia) was incorporated into the initiator paste of the commercially available temporary fixing cement based on polycarboxylate/zinc oxide Systemp.cem (Ivoclar Vivadent AG). The processing time (PT), setting time (ST) and Shore D hardness were then determined. The results are summarized in Table 2.

TABLE 2
Properties of temporary cements based
on polycarboxylate/zinc oxide

| | Shore D | | |
|---|---|---|---|
| Cement | hardness | PT (s) | ST (s) |
| Systemp.cem*) | 56 | 115 | 208 |
| Systemp.cem + 0.3 wt.-% Ia | 57 | 108 | 199 |
| Systemp.cem + 3.0 wt.-% Ia | 59 | 105 | 187 |

*)Comparison

As Table 2 shows, the addition of 0.3 or 3.0 wt.-% of active ingredient (Ia) does not have a significant negative influence on the hardening of Systemp.cem.

Systemp.cem is a two-component fixing cement for the temporary fixing of temporary crowns and bridges based on zinc oxide-polycarboxylate. The base paste consists of a mixture of zinc oxide, glycerol and water, the catalyst paste consists of a mixture of SiO₂ particles, polyacrylic acid, water and olive oil.

### Example 4

### Antimicrobial Composite-Based Temporary Fixing Cement

0.3 wt.-% or 3.0 wt.-% of the antimicrobial active ingredient of formula (Ia) was incorporated into the base paste of the commercially available composite-based temporary fixing cement Systemp.link (Ivoclar Vivadent AG). The processing time (PT), setting time (ST) and Shore D hardness were then determined. The results are summarized in Table 3.

TABLE 3
Properties of composite-based temporary cements

| | Shore D | | |
|---|---|---|---|
| Cement | hardness | PT (s) | ST (s) |
| Systemp.link*) | 40 | 258 | 117 |
| Systemp.link + 0.3 wt.-% Ia | 40 | 258 | 119 |
| Systemp.link + 3.0 wt.-% Ia | 42 | 262 | 116 |

*)Comparison

As Table 3 shows, the addition of 0.3 or 3.0 wt.-% of active ingredient (Ia) does not have a significant negative influence on the hardening of Systemp.link.

Systemp.link is a dual-curing two-component fixing cement for the temporary fixing of provisional crowns and bridges based on composite. The base paste consists of a mixture of SiO₂ particles, a splinter polymerisate, a monomer mixture and a tertiary aromatic amine. The catalyst paste consists of a mixture of SiO₂ particles, a splinter polymerisate, a monomer mixture, dibenzoyl peroxide and a camphorquinone/amine photoinitiator system.

### Example 5

### Antimicrobial Temporary Filling Material

0.2 wt.-% or 0.5 wt.-% of the antimicrobial active ingredient of formula (Ia) was incorporated into the commercially available composite-based temporary filling material Fermit (Ivoclar Vivadent AG). After 20 s exposure to light with a dental polymerization light (Bluephase®, Ivoclar Vivadent AG), the material hardened completely. The Shore D hardness was then determined (see Table 4 for results).

TABLE 4
Shore D hardness of temporary filling materials

| | Shore D |
|---|---|
| Filling material | hardness |
| Fermit*) | 43 |
| Fermit + 0.2 wt.-% Ia | 45 |
| Fermit + 0.5 wt.-% Ia | 44 |

*)Comparison

As Table 4 shows, the addition of 0.2 or 0.5 wt.-% of active ingredient (Ia) does not have a significant negative influence on the hardening of Fermit.

Fermit is a temporary single component filling material which can be hardened with blue light. The composition consists of a mixture of SiO₂ particles, a splinter polymerisate, a monomer mixture and a camphorquinone/amine photoinitiator system.

### Example 6

### Antimicrobial Coating Material

(i) 12.0 wt.-% methyl methacrylate;
(ii) 66.7 wt.-% di-pentaerythritol triacrylate;
(iii) 0.1 wt.-% of the antimicrobial active ingredient of formula (Ia);
(iv) 7.9 wt.-% SiO₂ nanoparticles;
(v) 11.5 wt.-% ethanol;
(vi) 1.0 wt.-% 2,4,6-trimethylbenzoyldiphenylphosphine oxide;
(vii) 0.3 wt.-% camphorquinone; and
(viii) 0.5 wt.-% of the co-initiator ethyl-p-dimethylaminobenzoate.

After 20 s exposure to light with a dental polymerization light (Bluephase®, Ivoclar Vivadent AG), the coating material hardened completely without oxygen inhibition.

### Example 7

### Antimicrobial Flowable Filling Material

A composite filling material with the following composition was prepared:

| Component | wt.-% |
|---|---|
| Binder¹⁾ | 16.8% |
| Filler²⁾ | 48.5% |
| Prepolymer | 34% |
| Additives, catalysts and stabilizers | 0.7% |
| Pigments | <0.1% |

¹⁾bis-GMA, urethane dimethacrylate, ethoxylated bis-EMA
²⁾barium glass filler, ytterbium trifluoride, mixed oxide

3.0 wt.-% or 5.0 wt.-% of the antimicrobial active ingredient of formula (Ia) was incorporated into the composite filling material. After 20 s exposure to light with a dental polymerization light (Bluephase®, Ivoclar Vivadent AG), the through-curing depth was determined. For comparison, the antimicrobial active ingredients triclosan and chlorhexidine diacetate were also incorporated into the composite material in a quantity of 3.0 wt.-% or 5.0 wt.-% and the through-curing depth determined (see Table 5 for results).

As Table 5 shows, the addition of active ingredient (Ia) does not have a significant negative influence on the through-curing depth of the composite material. On the other hand, the through-curing depth of the composite material is dramatically reduced by the addition of triclosan or chlorhexidine diacetate.

TABLE 5
Through-curing depth of light-curing composite filling materials

| | Through-curing |
|---|---|
| Composite filling material | depth (mm) |
| Composite material*) | 4.12 |
| Composite material + 3.0 wt.-% triclosan | 3.04 |
| Composite material + 5.0 wt.-% triclosan | 2.48 |
| Composite material + 3.0 wt.-% | 3.83 |
| chlorhexidine diacetate | |
| Composite material + 5.0 wt.-% | 3.17 |
| chlorhexidine diacetate | |
| Composite material + 3.0 wt.-% Ia | 4.13 |
| Composite material + 5.0 wt.-% Ia | 4.09 |

*)Comparison

### Example 8

### Influence of Triclosan and of the Active Ingredient of Formula (Ia) on the Radical Polymerization

3.0 wt.-% triclosan or 3.0 wt.-% of the antimicrobial active ingredient of formula (Ia) was incorporated into the base paste of the commercially available composite-based temporary fixing cement Systemp.link (Ivoclar Vivadent AG), which hardens radically. The processing time (PT), setting time (ST) and Shore D hardness in the case of chemical hardening were then determined. The results are summarized in Table 6.

TABLE 6
Properties of temporary fixing cements

| | Shore D | | |
|---|---|---|---|
| Temporary cement | hardness | PT (s) | ST (s) |
| Systemp.link*) | 40 | 258 | 117 |
| Systemp.link + 3.0 wt.-% triclosan*) | not | not | not |
| | polym. | polym. | polym. |
| Systemp.link + 3.0 wt.-% Ia | 41 | 260 | 115 |

*)Comparison

As Table 6 shows, the addition of active ingredient (Ia) does not have a significant negative influence on the hardening of Systemp.link. On the other hand, the chemical hardening of Systemp.link is completely inhibited by the addition of triclosan.

### Example 9

### Determination of the Antimicrobial Action of a Composite-Based Temporary Cement with the Active Ingredient According to Formula (Ia)

To determine the antimicrobial action of a temporary dental cement with the active ingredient of formula (Ia), 3.0 wt.-% of the active ingredient of formula (Ia) was incorporated into the commercially available cement Systemp.link (Ivoclar Vivadent AG). Round testpieces with a diameter of 10 mm and a thickness of 2 mm were then shaped into small plates with the corresponding material and then cured. Testpieces made of Systemp.link without active ingredient and also with 1.0 and 3.0 wt.-% triclosan served as reference. After this, the antimicrobial action on the bacterial strain Streptococcus mutans was studied. The agar plate zone of inhibition test was used for this.

Bacterial strain: S. mutans DSM20723
Cultivation medium: BHI broth

### Agar: BHI

100 μl undiluted S. mutans culture solution was plated out onto agar plates. The testpieces were then placed on the agar plates. After 24 h storage at 37° C./5% CO₂, the plates were assessed.

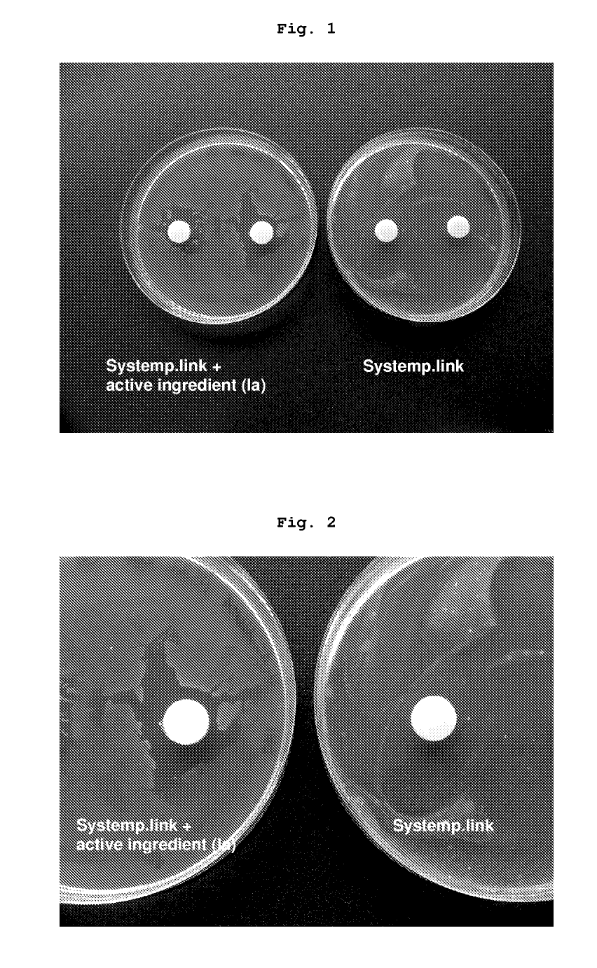

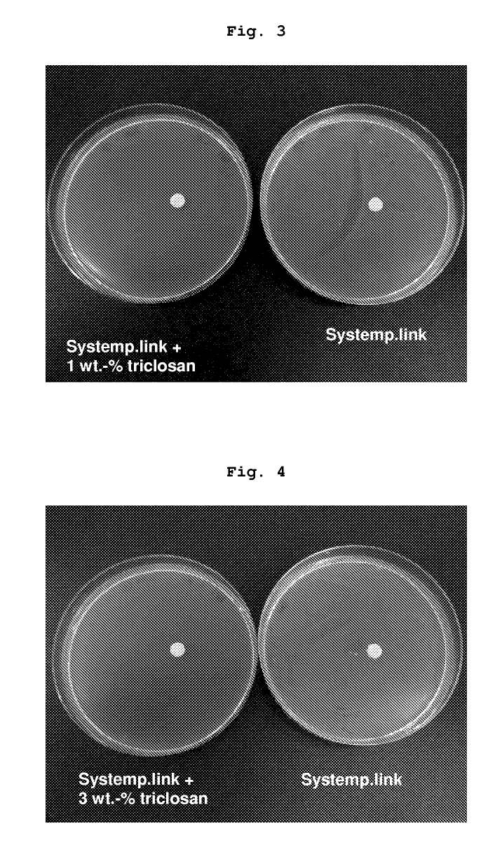

As can be seen in FIGS. 1 and 2, clear zones of inhibition can be seen in the case of the Systemp.link small plates (light-cured) with added active ingredient of formula (Ia). The reference sample, on the other hand, showed no inhibition of bacterial growth (no formation of zones of inhibition). Surprisingly, the addition of 1.0 or 3.0 wt.-% triclosan also does not bring about a formation of zones of inhibition (FIGS. 3 and 4). The test shows that the dental materials according to the invention release active ingredient of formula (I) in an effective quantity.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

1. Dental material comprising an antimicrobial active ingredient according to general formula (I), 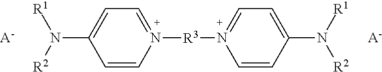
in which
R¹=H, C₁ to C₃ alkylene residue;
R²=a linear or branched C₄ to C₂₀ alkylene residue;
R³=a C₆ to C₂₀ alkylene residue; and
A⁻=Cl⁻, Br⁻, I⁻, F⁻, OH⁻, alkyl and aryl alcoholate, thiolate, NO₃⁻, BO₃³⁻, PO₄³⁻, HPO₄²⁻, H₂PO₄⁻, alkyl and aryl phosphate, alkyl and aryl phosphonate, PF₆⁻, TiF₆²⁻, BF₄⁻, acetylacetonate, β-diketonate or anion of a β-keto ester.

2. Dental material according to claim 1, which comprises an active ingredient according to formula (I), in which
R¹=H;
R²=a linear C₈ alkyl residue;
R³=a linear C₁₀ alkylene residue; and
A⁻=Cl⁻, alkyl phosphate, alkyl phosphonate.

3. Dental material according to claim 1, which comprises about 0.01-about 3.0 wt.-% of the active ingredient according to formula (I).

4. Dental material according to claim 1, which comprises about 0.05-about 1.5 wt.-% of the active ingredient according to formula (I).

5. Dental material according to claim 1, which comprises about 0.1-about 0.6 wt.-% of the active ingredient according to formula (I)

6. Dental material according to claim 1, which additionally comprises at least one polymerizable, ethylenically unsaturated monomer as binder and an initiator for the radical polymerization.

7. Dental material according to claim 1, which additionally comprises an organic and/or inorganic filler or a filler mixture.

8. Dental material according to claim 1, which comprises at least one monofunctional or polyfunctional (meth)acrylate or (meth)acryl amide or a mixture thereof as binder.

9. Dental material according to claim 1, which comprises a mixture of acidic and non-acidic monomers as binder.

10. Dental material according to claim 1, which additionally comprises a solvent.

11. Dental material according to claim 10, wherein the solvent comprises water, methanol, ethanol, phenoxyethanol, isopropanol, ethyl acetate, acetone or a mixture of two or more of the named solvents.

12. Dental material according to claim 1, which comprises
about 0.01-about 3.0 wt.-%, active ingredient according to formula (I);
about 20 to about 95 wt.-% binder;
about 0.1 to about 5 wt.-% initiator; and optionally
0 to about 90 wt.-% filler; and optionally
0 to about 80 wt.-% solvent.

13. Dental material according to claim 1, which comprises
about 0.05-about 1.5 wt.-% active ingredient according to formula (I);
about 30 to about 90 wt.-% binder,
about 0.3 to about 4.0 wt.-% initiator; and optionally
about 2 to about 85 wt.-% filler; and optionally
0 to about 60 wt.-% solvent.

14. Dental material according to claim 1, which comprises
about 0.1-about 0.6 wt.-% active ingredient according to formula (I);
about 35 to about 80 wt.-% binder,
about 0.5 to about 3.5 wt.-% initiator; and optionally
about 10 to about 80 wt.-% filler; and optionally
0 to about 40 wt.-% solvent.

15. Process of using the dental material according to claim 1 as tooth filling material, material for inlays or onlays, tooth cement, veneering material for crowns and bridges, or as material for false teeth, materials for temporary restorations or as other materials for prosthetic, restorative and preventative dentistry.

16. Process of using the dental material according to claim 1 as enamel/dentine adhesive.

17. Process of using the dental material according to claim 1 as coating material for tooth fillings, restoration materials, dentures and natural teeth.

18. Process of using the dental material according to claim 1 to prevent tooth damage caused by Streptococcus mutans and/or lactobacilli.

19. Process of using the dental material according to claim 1 to prevent prosthesis incompatibilities caused by Candida albicans.

20. Process of using an active ingredient according to formula (I) to prepare a polymerizable dental material wherein formula (I) comprises 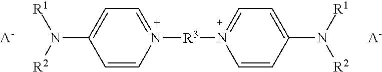
in which
R¹=H, C₁ to C₃ alkylene residue;
R²=a linear or branched C₄ to C₂₀ alkylene residue;
R³=a C₆ to C₂₀ alkylene residue; and
A⁻=Cl⁻, Br⁻, I⁻, F⁻, OH⁻, alkyl and aryl alcoholate, thiolate, NO₃⁻, BO₃³⁻, PO₄³⁻, HPO₄²⁻, H₂PO₄⁻, alkyl and aryl phosphate, alkyl and aryl phosphonate, PF₆⁻, TiF₆²⁻, BF₄⁻, acetylacetonate, β-diketonate or anion of a β-keto ester.
